(19) United States
(12) Patent Application Publication
(10) Pub. No.: US 2025/0359918 A1
(43) Pub. Date: Nov. 27, 2025

(54) METHODS AND APPARATUSES FOR TARGETED TUMOR-SPECIFIC ABLATION

(71) Applicant: Pulse Biosciences, Inc., Hayward, CA (US)

(72) Inventors: Richard L. NUCCITELLI, Hayward, CA (US); Esin B SÖZER, Hayward, CA (US)

(21) Appl. No.: 18/875,102

(22) PCT Filed: Jun. 14, 2023
(86) PCT No.: PCT/US2023/068462

§ 371 Date: Dec. 13, 2024

Related U.S. Application Data
(60) Provisional application No. 63/353,495, filed on Jun. 17, 2022.

(51) Int. Cl. A61B 18/12; A61B 17/00; A61B 18/00; A61B 18/14; G16H 40/63

(52) U.S. Cl. CPC A61B 18/1206; A61B 18/1477; G16H 40/63; A61B 2017/00172; A61B 2017/00185; A61B 2018/0016; A61B 2018/00577; A61B 2018/00732; A61B 2018/00761; A61B 2018/00875

(57) ABSTRACT

Methods and apparatuses for selectively treating, including killing, tissues or cells with packets of sub-microsecond duration, high frequency electrical pulses in which the packet size (e.g., number of pulses) delivered is set based on the membrane charging time constant for the target tissue or cells.

### CLAIM OF PRIORITY

This patent application claims priority to U.S. provisional patent application No. 63/353,495, titled “METHODS AND APPARATUSES FOR TARGETED TUMOR-SPECIFIC ABLATION,” filed on Jun. 17, 2022, herein incorporated by reference in its entirety.

### BACKGROUND

A targeted therapy is a type of cancer treatment that targets cancer cells, preferably without affecting normal cells. To date, most targeted cancer therapies are based on pharmacological, and in particular, immunological, treatments. Cancer cells typically have changes in their genes that make them different from normal cells. There are many different types of cancer, and not all cancer cells are the same. For example, colon cancer and breast cancer cells have different genetic changes that help them grow and/or spread. Targeted therapy is sometimes called precision medicine or personalized medicine. Target therapy is considered particularly beneficial because it may specifically target cancer cells, without harming non-cancer tissue.

Targeted therapy is proving to be an important type of cancer treatment, but to date only a few types of cancers are routinely treated using only these targeted drug therapies, and such therapies may still require surgery, chemotherapy, radiation therapy, or hormone therapy. Further it has proven difficult and expensive to identify and develop therapies that may effectively target cancer, and in particular tumor cells. This is particularly true for pharmaceutical targeted therapies.

What is needed are methods and apparatuses for specifically targeting different tissue types, and in particular tumor tissue, to treat a patient. The methods and apparatuses described herein may address these needs.

### SUMMARY OF THE DISCLOSURE

Described herein are methods and apparatuses for sub-microsecond energy application to selectively treat target tissues (e.g., tumor tissue) without substantially effecting non-target tissue, even when non-target tissue is coincident with the target tissue. In particular, described herein are methods and apparatuses for delivering sub-microsecond pulses at megahertz frequencies (e.g., 0.5 MHz or more), to provide time-constant based treatment (e.g., ablation) of a target tissue.

Sub-microsecond (e.g., nanopulse) treatment may be used to trigger regulated cell death in tissues, typically by applying about 50 monophasic, 200 ns-long pulses at electric fields of 25-30 kV/cm with 6-8 Hz repetition rate. Since the charging time constant of any typical tissue is much higher than the pulse duration of 200 ns and since the electric field amplitude is very high, the induced plasma membrane potential change during the pulse exposure will typically result in a similar membrane charging in virtually all cells. However, described herein are methods and apparatuses that may use sub-microsecond lengths pulses applied at very high repetition rates, e.g., pulsed within the MHz range (e.g., 0.5 MHz or more), and may be used in packets of, e.g., 50-300 pulses of 100 ns duration at a lower electric field amplitude, such as about 8 kV/cm but with a million times faster rate (typically 0.5 MHz-5 MHz). Packets may be repeated (e.g., at between about 1-5 Hz). These high-repetition rate pulse packets may have high duty cycles such that the time between individual 100 ns pulses is not enough for complete discharge of the induced membrane potential in most cells and tissues. This may allow membrane charging to continue through the duration of the whole packet (e.g., between 17-100 μs) and may result in an induced membrane charging reaching a maximum level at the end of the packet duration. As described herein, this may result in different tumor types with different cellular and extracellular compositions requiring different packet sizes for full ablation and may similarly result in targeting of tumor vs. non-tumor tissue due to different packet size thresholds since their electrical properties are expected to be different.

Thus, the methods and apparatuses described herein may deliver a fine-tuned electrical exposure that targets a specific tissue type (e.g., a tumor vs. non-tumor tissue) and can be useful when there is a mixture of electrically distinct cell and tissue types in the treatment area. The same principle can apply to any pathology that creates a local environment electrically distinct from its surroundings. Thus, described herein are apparatuses that may target specific tissue types based on the electrical time constant of the target vs. non-target tissue. Moreover, the methods and apparatuses, which may use megahertz frequencies of sub-microsecond pulses as described herein, may result in ablation at lower electric fields than traditional nanosecond pulsed electric fields of a typical frequencies of 1-10 Hz, while still inducing potentials on intracellular membranes with its high-frequency, sub-microsecond duration oscillations, which may induce intracellular stress to organelles such as the mitochondria and endoplasmic reticulum.

In general, the methods and apparatuses described herein may apply sub-microsecond pulses at megahertz frequencies, which may be used to provide time-constant-based tuning of packet sizes to target tumor tissue and spare surrounding normal tissue, as tumor tissues may have a lower time constant than their normal counterparts. Without being bound by a particular theory, this may be because tumor tissues have higher water content, lower cell volume fraction, and higher conductivity than normal tissue. Tumor tissues are also known to consist of cells with lower membrane potential compared to those within corresponding healthy tissue. The lower membrane potential may make it harder for cells in the tumor to recover the ionic concentrations needed for cellular homeostasis, making it less likely for those cells to survive after electric field exposure. Tissue also has a different time constant than dissociated cells.

In general, the methods and apparatuses described herein may select or set the sub-microsecond energy application to selectively treat target tissues (e.g., tumor tissue) or cells without substantially affecting non-target tissue or cells based on the membrane charging time constant for the target tissue or cells. The membrane charging time constant may be determined directly or indirectly. For example, the membrane charging time constant may be determined by direct measurement, either from the target tissue or a biopsy of the target tissue (including a cultured or transplanted biopsy), or it may be determined, for example, directly by voltage/current measurement following the application of a low-energy electric pulse, or by indirect measurement from the target tissue or a biopsy of the target tissue, e.g., by determining a characteristic property that is correlated with the membrane charging time constant, such as the bioimpedance, fat content, water content, etc. In some examples the membrane charging time constant may be determined by identifying the size, shape and/or type of cells (either isolated cells or tissue), including based on identifying the type of tumor, and using a database of known or approximate membrane charging time constants associated with the particular type of target tissue and/or property of the target tissue.

As used herein, the membrane charging time constant may be referred to as the membrane time constant, the charging time constant or simply the time constant. The membrane charging time constant is generally the same as the membrane discharging time constant. The membrane time constant is a function of two properties of membranes of the cell/tissue, the membrane resistance (Rₘ) and the membrane capacitance (Cₘ) (Rₘ is inversely related to permeability). Membrane time constants may be empirically determined by measuring the electrical response of a tissue or cell, or they may be estimated, as described herein.

As used herein, the term “packets” refers to a group or burst of sub-microsecond (e.g., nanosecond) pulses within the MHz range (e.g., 0.5 MHz or greater, between about 0.5 MHz and 10 MHz, 0.7 MHz and 5 MHz, etc.). The packet size refers to the duration of the packet (e.g., burst length), in time (e.g., microsecond duration or longer). Packets may be repeated for treatment, e.g., at a frequency of between about 0.1 Hz and about 10 Hz, between about 1 Hz and 10 Hz, between about 1 Hz and 5 Hz, etc.).

In general, these methods and apparatuses may be used to selectively treat (e.g., kill or ablate) all or most of a target tissue or cells. The target tissue or cells may generally be rapidly dividing cells or tissue comprising rapidly-dividing cells, which may generally have a membrane charging time constant that is faster than the membrane charging time constants of more slowly dividing cells, including the same original tissue/cell type giving rise to a tumor. In some examples the target tissue or cells is a target tissue that is a tumor; in some examples the target tissue comprises a cancerous (e.g., malignant) tumor. In general, these methods and apparatuses may distinguish between tissue types based on the membrane charging time constant and may use the time constant to set the treatment parameters, including in particular the number of pulses delivered over time (e.g., the packet size) so that only or primarily the target tissue or cells (e.g., tumor tissue or cells) are treated, while non-target tissue, even when coincident with the target tissue, is not significantly treated. As mentioned, treatment may include killing the tissue or cells, including inducing regulated cell death.

For example, described herein are methods of specifically killing a target tissue or cells, the method comprising: setting a packet size of a packet of sub-microsecond pulsed electrical energy having a frequency of greater than 0.5 MHz (e.g., 0.75 MHz, 1 MHz, etc.) based on an identified membrane charging time constant for the target tissue or cells; positioning the target tissue or cells between two or more electrodes; and killing at least some of the target tissue or cells by applying the packet of sub-microsecond pulsed electrical energy between the two or more electrodes.

For example, a method of specifically ablating a target tissue may include: setting a packet size of a packet of sub-microsecond pulsed electrical energy having a frequency of between 0.5 MHz and 5 MHz wherein the packet size is between about 3 or more times a membrane charging time constant for the target tissue and about 2 or less times a membrane charging time constant for a non-target tissue or cells; positioning the target tissue between two or more electrodes; and killing at least some of the target tissue by applying the packet of sub-microsecond pulsed electrical energy between the two or more electrodes, wherein a non-target issue between the two or more electrodes is not killed.

In some examples, the method may include a method of specifically ablating a target tissue, such method comprising: setting a packet size of a packet of sub-microsecond pulsed electrical energy having a frequency of between 0.5 MHz and 5 MHz based on the identified membrane charging time constant for the target tissue; positioning the target tissue between two or more electrodes; and inducing regulated cell death at least in some of the target tissue by applying the packet of sub-microsecond pulsed electrical energy between the two or more electrodes, wherein a non-target issue between the two or more electrodes is not killed. The method may further include identifying a membrane charging time constant for the target tissue.

Any of these methods may include identifying a membrane charging time constant for the target tissue or cells. For example, identifying the membrane charging time constant may comprise receiving a description of the target tissue or cells and looking up the membrane charging time constant based on the description. For example, identifying the membrane charging time constant may comprise determining a bioimpedance measurement from the target tissue or cells and estimating the membrane charging time constant for the target tissue from the bioimpedance measurement. In some examples identifying the membrane charging time constant may comprise determining a fat and/or water content of the target tissue or cells and estimating the membrane charging time constant from the fat and/or water content. In some examples identifying the membrane charging time may comprise receiving a description of the target tissue or cells comprising one or more of: a tissue type of the target tissue or cells, a cell size of the target tissue or cells, a cell shape of the target tissue or cells, a fat content of the target tissue or cells, and a water content of the target tissue or cells, and determining the membrane charging time constant based on the description. In some examples identifying the membrane charging time constant comprises using a low-energy test pulse (e.g., low voltage, microsecond or longer duration) to measure voltage/current waveforms to determine a time constant from the resulting electrical properties, such as the time course of the conductance following the test pulse.

In general, the methods and apparatuses may select and may fine-tune the packet size based on the treatment-specific variations in cell size, cell shape, fat/water content. In some cases, bioimpedance may be used to determine the membrane charging potential, either directly or by determining the fat and/or water content of the tissue or cells. For example, determination of variation in fat/water content can be done with low-voltage bioimpedance measurements. A higher fat ratio may lead to a lower conductivity of the tissue, which will then translate into a higher time constant and larger packet size.

Alternatively or additionally, in some examples identifying the membrane charging time constant comprises estimating the membrane charging time constant from a biopsy of the target tissue. For example, identifying the membrane charging time constant may include estimating the membrane charging time constant from a biopsy of the target tissue grown outside of a patient from whom the biopsy was taken. In some examples, packet size can be customized by pre-treatment estimation of the time constant, including (but not limited to) determining membrane charging time constant for target tissue based on sub-microsecond pulse MHz electric field ablation of a tumor grown on nude mice by injection of tumor cells cultured from the biopsy of the patient. In some cases, the packet size can be customized by pre-treatment estimation of the time constant based on current measurements of ex-vivo normal tissue to exposure to varying package sizes to find the threshold to spare normal tissue.

In general, setting the packet size of the packet of sub-microsecond pulsed electrical energy may include setting the packet size based on the membrane time charging constant for the target tissue or cells; in some examples, this may alternatively or additionally include setting the packet size of the packet of sub-microsecond pulsed electrical energy based on the membrane charging time constant of the non-target cells. For example, the size of the packet may be set so that the generally faster membrane charging time constant tissue or cells treated (e.g., killed) while the generally slow membrane charging time constant non-target tissues or cells are not treated (e.g., not killed). The non-target tissues or cells may not be treated (e.g., killed) because the membrane potential may fail to sum to the level necessary to treat the tissue or cells.

For example, setting the packet size of the packet of sub-microsecond pulsed electrical energy may comprise limiting or setting the packet size, for example, to 3 or more times the membrane charging time constant for the target tissue or cells. In some examples setting the packet size of the packet of sub-microsecond pulsed electrical energy comprises limiting the packet size to between about 3 or more times the membrane charging time constant for the target tissue and 2 or less times than a membrane charging time constant for a non-target tissue or cells between the two or more electrodes, where the membrane charging time constant for the target tissue or cells is less than the membrane charging time constant for the adjacent non-target tissue or cells (e.g., when 3 times the membrane charging time constant for the target tissue is less than twice, or in some cases three times, the membrane charging time constant for the adjacent non-target tissue). In the specific context in which the target tissue or cells are rapidly growing, it is usually the case that the target tissue or cells have a time constant that is sufficiently less than the membrane charging time constant for the non-target tissue.

As mentioned, in some examples the methods described herein may treat the target tissues or cells, but not significantly treat non-target tissues or cells, by killing the target tissue or cells which are between the two or more electrodes without killing a non-target issue or cells between the two or more electrodes. Treatment (e.g., killing) may refer to treating or killing a significant amount, e.g., 35% or more, 40% or more, 45% or more, 50% or more, 55% or more, 60% or more, 70% or more, 75% or more, a majority of the tissue or cells (e.g., cells of the target tissue), etc. For example, killing at least some of the target tissue or cells may comprise killing more than half of the target tissue or cells. In some examples killing comprises killing at least half of the target tissue or cells between the two or more electrodes without killing more than 40% of the non-target tissue between the two or more electrodes (e.g., 40% or less, 35% or less, 30% or less, 25% or less, 20% or less, 15% or less, 10% or less, 5% or less, etc.). In any of these examples, killing at least some of the target tissue or cells may include inducing regulated cell death in the target tissue or cells.

Any of these methods and apparatuses may include setting the parameters for delivering a packet of sub-microsecond pulsed electrical energy in which the sub-microsecond pulses have a pulse duration of between about 1 ns to about 1000 ns (e.g., nanosecond range pulses). The packet of sub-microsecond pulsed electrical energy may include sub-microsecond pulses having a pulse amplitude of about 1 kV/cm or more (e.g., 12 kV/cm or less, 10 kV/cm or less, 9 kV/cm or less, 8 kV/cm or less, etc.). Setting the packet size may comprise setting the packet between about 30 and 300 pulses; the actual packet size may be determined based on the actual or estimated membrane charging time constant, as described herein. The packet size may be based on the time (e.g., by determining the number of sub-microsecond pulses at the applied MHz rate within the packet).

Any appropriate target tissue may be used. For example, the target tissue may comprise a tumor (e.g., a malignant tumor, a cancerous tumor, etc.). In any of these examples the target tissue may comprise a tissue having rapidly dividing cells; the rapidly dividing cells may divide at a rate that is greater than normal health cells of the same original cell type (e.g., when treating tumor cells derived from skin tissue, the target tissue may have cells that divide more rapidly (e.g., greater than 1.5 fold faster, greater than 2 fold faster, greater than 2.5 fold faster, etc.).

In any of these methods and apparatuses, the applicator(s), e.g., electrode(s) may be positioned on either sides, or generally within, the target tissue. For example, positioning the target tissue or cells between two or more electrodes may comprise inserting two or more needle electrodes into a tissue proximal to the target tissue. In some examples, positioning the target tissue or cells between two or more electrodes may comprise placing the two more electrodes against a patient's skin, tissue or organ.

Also described herein are apparatuses for performing any of these methods, and apparatuses for treating target tissue while generally avoiding treating non-target tissue that may be concurrently positioned withing the body. According to one aspect, an apparatus for specifically ablating a target tissue is provided. The apparatus may comprise: a pulse generator; an applicator configured to apply electrical energy from the pulse generator to two or more electrodes; and a controller comprising one or more processors and a memory storing computer-program instructions, that, when executed by the one or more processors, perform a computer-implemented method comprising: setting a packet size of a packet of sub-microsecond pulsed electrical energy having a frequency of greater than 0.5 MHz based on the identified membrane charging time constant for the target tissue or cells; and applying, from the pulse generator, the packet of sub-microsecond pulsed electrical energy between the two or more electrodes, when triggered by a user input, to selectively and specifically treat the target tissue.

Any of these apparatuses may be configured, e.g., as part of the computer-program instructions, to identify the membrane charging time constant for the target tissue or cells and/or the non-target tissue or cells. The apparatus may guide the user through this process using one or more user interfaces. For example, a computer-implemented method may include receiving a description of the target tissue or cells and identifying a membrane charging time constant for the target tissue or cells. In some examples, identifying the membrane charging time constant comprises identifying the membrane charging time constant for the target tissue or cells based on the description. Receiving the description may include receiving one or more of: a tissue type of the target tissue or cells, a cell size of the target tissue or cells, a cell shape of the target tissue or cells, a fat content of the target tissue or cells, and a water content of the target tissue or cells. In some examples the computer-implemented method is configured to identify the membrane charging time constant by determining a fat and/or water content of the target tissue or cells and estimating the membrane charging time constant from the fat and/or water content.

The controller may be further configured to determine a bioimpedance measurement from two or more electrodes and wherein the computer-program instructions, further comprise identifying the membrane charging time constant for the target tissue or cells from the bioimpedance measurement.

In some examples the computer-implemented method (and/or an apparatus performing this method) is configured to set the packet size of the packet of sub-microsecond pulsed electrical energy by limiting the packet size, for example, to about 3 or more times the membrane charging time constant of the target tissue or cell, and/or setting the packet size to about 3 times or more than the membrane charging time constant for the target tissue or cells. In any of these methods and apparatuses the packet size may be set to between about 3 times the membrane charging time constant of the target tissue and about 2 times the membrane charging time constant of a non-target tissue or cell that is adjacent to (or equivalently intermixed with) the target tissue.

The apparatus may include the electrodes. For example, the apparatus may include a removable tip containing two or more electrodes that may attach (electrically and/or mechanically) to the applicator. In some examples, the two or more electrodes may comprise an array of penetrating electrodes (e.g., needle electrodes) or non-penetrating electrodes (e.g., surface electrodes).

In any of these apparatuses the computer-implemented method may be configured to set the packet size of the packet of sub-microsecond pulsed electrical energy so that the sub-microsecond pulsed electrical energy comprises sub-microsecond pulses having a pulse duration of between about 1 ns to about 1000 ns. The computer-implemented method may be configured to set the packet size of the packet of sub-microsecond pulsed electrical energy so that the sub-microsecond pulsed electrical energy comprises sub-microsecond pulses having a pulse amplitude of between about 1 kV/cm to about 12 kV/cm. The computer-implemented method may be configured to set the packet size of the packet of sub-microsecond pulsed electrical energy so that the packet size comprises between about 30 and 300 pulses.

In some examples, the methods and apparatuses described herein for sub-microsecond energy application to selectively treat target tissues may provide similar plasma membrane charging as an equivalent microsecond or longer pulses but may be significantly more efficient in eliminating tumors. Thus, microsecond-long pulse exposures with equivalent capacitive charging as the sub-microsecond, MHz packets described herein are significantly less efficient in tumor clearance. This may indicate the additional biological benefit of high-frequency components of nanosecond duration pulses, which may cause sustained oscillations in intracellular membranes, unlike their microsecond pulse counterparts. Further, these methods and apparatuses using sub-microsecond, MHz pulse packets may specifically treat target tissue while having little or no effect on non-target tissue.

In general, any of these apparatuses may include a controller that is configured to identify the membrane charging time constant by applying a low-energy test pulse and determining a time constant from a time course of a conductance from the test pulse, as described herein.

Any of these apparatuses may be configured as apparatuses for specifically ablating a target tissue. The apparatus may include: a pulse generator; an applicator configured to apply electrical energy from the pulse generator to two or more electrodes; and a controller comprising one or more processors and a memory storing computer-program instructions, that, when executed by the one or more processors, perform a computer-implemented method comprising: identifying a membrane charging time constant for the target tissue; setting a packet size of a packet of sub-microsecond pulsed electrical energy having a frequency of between 0.5 MHz and 5 MHz based on the identified membrane charging time constant; and applying, from the applicator, the packet of sub-microsecond pulsed electrical energy between the two or more electrodes to selectively and specifically kill the target tissue.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates megahertz compression or summation of sub-microsecond pulsed electrical fields illustrated using 10 pulse packet of 3 MHz repetition rate train, and its induced potential on a membrane with charging time constant of 1 μs.

FIGS. 2A-2D show examples of transillumination views of murine melanoma in skin stretched over a light source. Needle holes made by treating electrode can be seen in the Post Tx image. The tumor shrinks slowly and is nearly gone by 25 days post Tx.

FIG. 3 is a graph illustrating the percentage of mice exhibiting 100% B16 melanoma tumor ablation following treatment with the indicated energy of either traditional, 8 Hz of sub-microsecond pulses or 1000 pulse packets of 3 MHz of sub-microsecond pulses.

FIGS. 4A-4B are graphs showing complete ablation percentage of B16 tumors using 3 MHz pulse exposures having either packets of 50 pulses or packets of 100 pulses repeated for different numbers of times (e.g., at between about 3 Hz). The energy applied (e.g., in FIG. 4A, 5.3 J, 11 J or 16 J, in FIG. 4B 11 J and 21 J) therefore depended on the number of times the packet was repeatedly delivered. In FIGS. 4A and 4B, 50 pulse (50 p) packets repeated for more than 100 repetitions and 100 pulse (100 p) packets repeated for 50 times both successfully ablate the tumors at 11 J. Data from two different experiments, numbers of animals for each condition are indicated in the figures.

FIG. 4C is a box plot comparing packets of 50 or 100 pulses each delivered to B16 tumor tissue at 11 J.

FIG. 4D is a graph showing the successful ablation of B16 tumor tissue in one experiment using sub-microsecond pulses applied in 3 MHz packets of 100 pulses (4 kV pulses) each, repeated 50 times (e.g., 10.6 J).

FIGS. 5A and 5B illustrate examples of the percentage of complete ablation of SCC7 tumors using packets of 3 MHz, sub-microsecond pulses with either 100 pulse packets (FIG. 5A) or 150 pulse packets (FIG. 5B) repeated for different numbers of times (e.g., at 3 Hz). 100 p packets were not successful in complete ablation up to 50 J, while 150 p packets showed higher success rate than 100 p packets even at 16 J. Data from five different experiments, numbers of animals for each condition are indicated on the figures.

FIGS. 5C and 5D are box plots comparing the percent complete clearance (ablation) of SCC7 tumors based on packet size for equivalent applied energies (32 J in FIG. 5C, 43-48 J in FIG. 5D). Effective clearance of SCC7 tumors was achieved with packets of 150 pulses or more using sub-microsecond pulses at 3 MHz.

FIG. 5E shows a box plot comparing the percent complete clearance (ablation) of LCC tumors based on packet size for equivalent applied energies (43 J) at different packet sizes (e.g., 100, 150 or 200 pulses).

FIG. 6 is a graph showing a theoretical calculation of plasma membrane charging with B16 and SCC7 tumors based on complete ablation results obtained with 3 MHz packets of different sizes shown in FIGS. 4A-4D and FIGS. 5A-5D.

FIGS. 7A-7C show the results of SCC7 tumor ablation as described herein, using packets of sub-microsecond, 3 MHz pulses each having 100 pulses applied for different repetitions of pulses (different exposures). FIG. 7A shows the change in tumor volume over time for 26.6 J (e.g., 125 repeated packets of 4 kV pulses repeated at 3 Hz), FIG. 7B shows the change in tumor volume over time for 32 J (e.g., 150 repeated packets at 3 Hz) and FIG. 7C shows the change in tumor volume over time for 42.6 J (e.g., 200 repeated packets at 3 Hz). In all of these examples the SCC7 tumors did not respond to 100 pulse packets at any of the energy levels applied.

FIG. 7D-7F show the results of SCC7 tumor ablation similar to that shown in FIGS. 7A-7C, using packets of sub-microsecond, 3 MHz pulses each having 150 pulses applied for different voltages or repetitions of pulses. FIG. 7D shows the change in tumor volume over time for 12 J (e.g., 50 packets of 3.5 kV pulses repeated at 3 Hz), FIG. 7E shows the change in tumor volume over time for 16 J (e.g., 50 packets of 4 kV pulses repeated at 3 Hz) and FIG. 7F shows the change in tumor volume over time for 47 J (e.g., 150 packets of 4 kV pulses repeated at 3 Hz). In contrast to FIGS. 7A-7C, all of these examples the SCC7 tumors respond well to 150 pulse packets with as low as 12 J.

FIGS. 8A-8B are graphs illustrating charging of cellular membranes exposed to sub-microsecond pulsing at high (e.g., megahertz) frequencies (100 ns, 3 MHz, 50-pulse packet), an equivalent-charging microsecond pulsing (17 μs, amplitude is scaled by the duty cycle of the 3 MHz train), and a more traditional, low repetition rate sub-microsecond pulse (200 ns). FIG. 8A shows plasma membrane charging. FIG. 8B shows intracellular membrane charging. As shown, intracellular membrane charging is significantly different between the three.

FIGS. 9A-9B are bar graphs comparing SCC7 tumor ablation (clearance) between packets of sub-microsecond, 3 MHz pulses at different numbers of packet repetitions (e.g., 100, 150) and microsecond-duration pulses applied for equivalent cellular membrane charging. FIG. 9A shows a comparison between repeated application of 50 μs pulses and packets of 150 sub-microsecond pulses at 3 MHz at 100 or 150 repetitions. FIG. 9B shows a comparison between the repeated application of 67 μs pulses and packets of 200 sub-microsecond pulses at 3 MHz at 100 repetitions. Error bars are +/−SEM.

FIGS. 9C and 9D are bar graphs comparing LLC tumor ablation (clearance) between packets of sub-microsecond, 3 MHz pulses at different numbers of packet repetitions and microsecond-duration pulses applied for equivalent cellular membrane charging. FIG. 9C shows a comparison between repeated application of 50 μs pulses and packets of 150 sub-microsecond pulses at 3 MHz at 135 or 150 repetitions. FIG. 9D shows a comparison between the repeated application of 67 μs pulses and packets of 200 sub-microsecond pulses at 3 MHz at 100 repetitions. Error bars are +/−SEM.

FIGS. 10A-10C illustrate examples of conductance plots of LLC (FIG. 10A), SCC7 (FIG. 10B), and B16 (FIG. 10C) tumors with μs-length pulse exposures.

FIGS. 10D and 10E illustrate one technique for estimating charging time constant using the pulses. FIG. 10D shows conductance curves of μs-length pulse exposure treatments (from FIGS. 10A-10C), to estimate the time constant of different tumor types and consequently estimate the ideal packet size for treatments. FIG. 10D shows scaled conductance curves for LLC, SCC7, and B16 tumors. FIG. 10E shows time constant fits for the first 25 μs after the peak of the conductance curves.

FIG. 11 is a schematic example of an apparatus for treating patients using the method as described herein (e.g., sub-microsecond pulsing at high, e.g., megahertz range frequencies in which the membrane charging constant may be determined or input and the system may use the target tissue or cells).

### DETAILED DESCRIPTION

Described herein are methods and apparatuses for selectively treating tissues or cells with packets of sub-microsecond duration, high frequency electrical pulses in which the packet size (e.g., number of pulses) delivered is set based on the membrane charging time constant for the target tissue or cells. These methods and apparatuses (e.g., devices, systems, etc.) may therefore effect target tissues or cells specifically, without significantly treating non-target tissues or cells that are adjacent or even commingled with the target tissue or cells. Thus, the methods and apparatuses described herein may distinguish between tissue types (e.g., target and non-target tissues) based on the membrane charging time constant. Any of these method and apparatuses may also identify the membrane charging time constant for the target (and in some cases the non-target) tissue and may set or adjust the electrical treatment parameters according to the identified membrane charging time constant(s). In addition to the highly specific targeting of target vs. not-target tissues or cells, these methods and apparatuses may also result in significantly lower energy densities needed to effectively treat the target tissues or cells.

The application of pulses of electrical energy to tissues, including tumor tissues, may result in charging the membrane potential of the tissue. Tissues have a characteristic membrane charging time constant, τ. The application of very rapid pulses to a tissue being treated may result in charging of the tissue when the pulse is on, while the tissue may discharge when the pulse is off. As described herein, sub-microsecond pulses (e.g., nanosecond duration pulses) may be delivered in packets that may be specifically tailored to for a particular target tissue within a patient, including a particular tumor tissue. Although the individual sub-microsecond pulses may have a duration that is much less than the membrane charging time constant for the target (and any non-target) tissue, when the sub-microsecond pulses are applied at a sufficiently high frequency (e.g., 0.5 MHz or greater, 0.75 MHz or greater, 1 MHz or greater, 1.5 MHz or greater, 2 MHz or greater, 2.5 MHz or greater, 3 MHz or greater, 3.5 MHz or greater, 4 MHz or greater, between 0.5-5 MHz, between 1-3 MHz, etc.) the number of sub-microsecond pulses applied as a packet (burst) of pulses may be chosen based on the specific membrane potential charging time constant for the target tissue in order to allow for capacitive summation of the pulses at the target membrane(s) of the target tissue/cells during treatment. At the same time, non-target tissues, which typically have longer membrane charging time constants, will not be significantly treated, if at all. Although all of the tissue and cells (both target and non-target) within a treatment area may be exposed to the application of electrical energy, only tissue or cells having membrane charging time constants matching (or shorter than) the applied packet size will be significantly treated. This permits the systems and apparatuses described herein to selectively treat just a subpopulation of the cells (or tissues including these cells), while leaving non-target tissues relatively intact.

As used herein, the megahertz (MHz) range may refer to 0.5 MHz or more (e.g., 0.75 MHz or more, 0.8 MHz or more, 1 MHz or more, 1.1 MHz or more, etc.).

In particular, these methods and apparatuses may adapt and simplify the procedure for the user. It is unlikely that a particular user would know, a priori, what the membrane charging time constant of a particular target tissue or cell type is, the methods and apparatuses may be configured to determine the charging time constant, and/or a range of pulse properties, including but not limited to number of pulses (e.g., packet size) appropriate for the target tissue/cells based on a determined charging time constant.

As used herein, treating may refer to killing (which should be understood to broadly include ablating, inducing apoptosis, inducing regulated cell death, etc.) of the tissue or cells. For example, devices, systems and methods described herein may be utilized in various ablation procedures (e.g., cancer treatments), dermatological procedures (e.g., treating various dermatological conditions, such as skin cancers), general surgery procedures (e.g., pancreatectomy), cardiology (e.g., valve repair), gynecology (e.g., hysterectomy), neurosurgery (e.g., tumor resection) etc. The methods and apparatuses described herein may also or alternatively be applied to excitable tissues (including but not limited to neuronal tissues) for either excitation and/or ablation treatments. For example, the methods and apparatuses described herein may be used for the stimulation of excitable tissues such as nerve and heart muscle (e.g., to treat neurological disorders such as epilepsy, Parkinson's disease and stroke). Heart disorders could include atrial fibrillation and ventricle fibrillation. The membrane potential of one or a group of cells may be excited directly using the methods described herein. The methods and apparatuses described herein may be used to stimulate secretion in cells (such as, but not limited to platelets). The methods and apparatuses described herein may treat tissues or cells of the brain, peripheral nerves, muscles, and heart. These methods and apparatuses may be used to treat any indication in which it may be beneficial to modulate or introduce action potentials (AP) in nerve and/or muscle targets. Alternatively or additionally, any of the methods and apparatuses described herein may be used for electroporation.

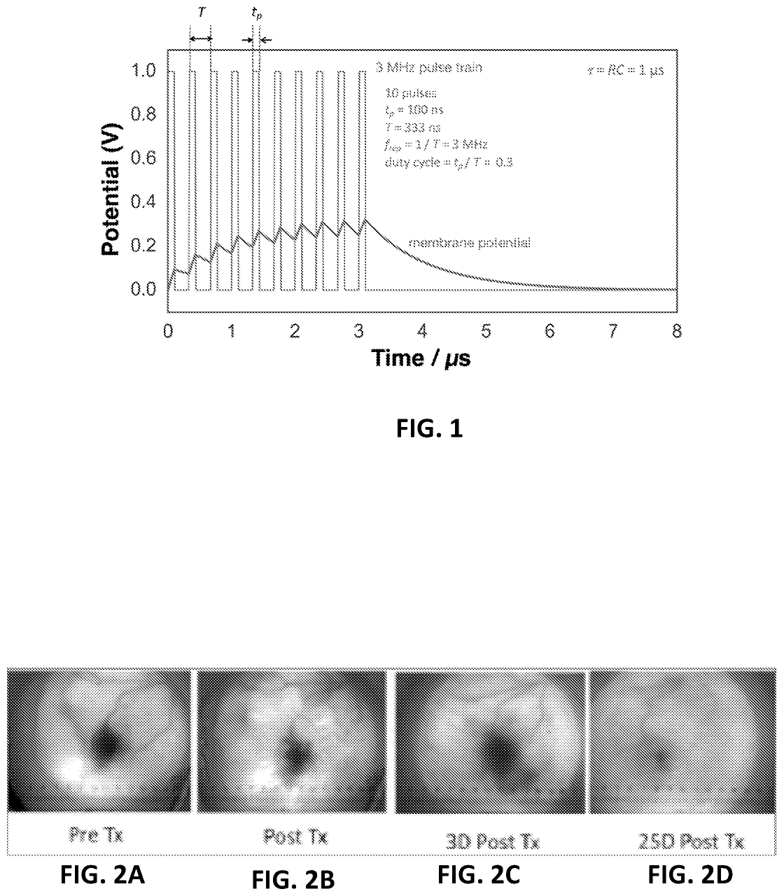

In general, these methods and apparatuses may be used to apply bursts (packets) of very short (sub-microsecond, e.g., nanosecond), pulses at high frequencies (e.g., 0.5 MHz or greater). For example, FIG. 1 shows an example of a packet of 10 pulses of 100 ns duration having a frequency of 3 MHz (resulting in a duty cycle of 0.3). FIG. 1 also shows an example of a packet applied to a cell having membrane charging time constant that is approximately 1 μs, showing the summation of the pulses to a plateau. The charging of tissue and cells may be electrically modeled as an RC circuit.

Thus, in general, the apparatuses and methods described herein may be configured to apply very brief (sub-microsecond, e.g., nanosecond) pulses within a packet having of high frequency (e.g., 0.5 MHz or greater, 0.75 MHz or greater, 1 MHz or greater, 3 MHz or greater, etc.). Either cells or tissue (or both) may be treated. In particular, tissues may be treated and tissues that have rapidly dividing cells, such as tumor tissues, including cancerous or malignant tumors, may be preferentially targeted, as the membrane charging time constant may be significantly lower for these rapidly dividing cells as compared to nearby non-target cells. Although all of the tissue between the electrodes (both target and non-target) may be charged by the application of the energy described herein, by limiting the size of the packet, and therefore the total number of pulses applied at high (e.g., 0.5 MHz or greater) frequency, only target tissues or cells having a sufficiently low time constant will be treated. For example, when the packet size is greater than about 2 or 3 times the membrane charging time constant of the target tissue, the target tissue will be treated. Cells or tissue with longer time constants will not be treated. Thus, the packet size is selected so that the size of the packet (e.g., in units of time, such as microseconds) is greater than between about 2 and 3 times the charging time constant of the target tissue or cells. In some examples the packet size may also be selected so that it is less than about 2 to 3 times the charging time constant of the nearby non-target cells or tissue. The size of the packet may also be described in terms of the number of pulses, such as the number of pulses of a given pulse width (e.g., less than 1 μs, such as 100 ns) at the MHz frequency (e.g., 0.5 MHz or greater). In general, the packet size may be greater than 1× the membrane charging time constant of the target cells (e.g., greater than 1.5× the membrane charging time constant of the target cells or tissue, greater than 1.8× the membrane charging time constant of the target cells or tissue, greater than 2× the membrane charging time constant of the target cells or tissue, greater than 2.2× the membrane charging time constant of the target cells or tissue, greater than 2.5× the membrane charging time constant of the target cells or tissue, greater than 2.8× the membrane charging time constant of the target cells or tissue, greater than 3× the membrane charging time constant of the target cells or tissue, greater than 3.2× the membrane charging time constant of the target cells or tissue, greater than 3.5× the membrane charging time constant of the target cells or tissue, etc.). Similarly, when the target tissue or cells has a smaller (faster) membrane charging time constant than non-target tissue, the packet size may also be selected so that it is less than an appropriate multiple membrane charging time constant of the non-target tissue or cells (e.g., less than 1× the membrane charging time constant of the non-target cells or tissue, less than 1.5× the membrane charging time constant of the non-target cells or tissue, less than 1.8× the membrane charging time constant of the non-target cells or tissue, less than 2× the membrane charging time constant of the non-target cells or tissue, less than 2.2× the membrane charging time constant of the non-target cells or tissue, less than 2.5× the membrane charging time constant of the non-target cells or tissue, less than 2.7× the membrane charging time constant of the non-target cells or tissue, less than 3× the membrane charging time constant of the non-target cells or tissue, etc.), where the higher the multiple, the less likely some percentage of the non-target tissue or cells will be treated.

In practice, the voltage necessary to treat a tissue or cells may be less than the voltage applied when treating with other sub-microsecond pulse trains, and may be optimized based on the tissue.

Most tissues have their own membrane charging time constant that may depend, at least in part, on the size of the cells within the tissue. In general tissues may have significantly longer time constants as compared to single cells, and faster-dividing (e.g., tumor) cells and tissues may have a significantly shorter membrane charging time constant as compared to even the parent cells or tissue type that gave rise to the tumor. The methods and apparatuses described herein may take advantage of these differences in the membrane charging time constants to specifically treat target cells and/or tissue. These methods may tune the packet size (and therefore the number of pulses applied at relatively high frequencies, e.g., between 0.5-10 MHz, between 0.75-6 MHz, between 1-5 MHz, between 1-4 MHz, between 1-3 MHz, etc.) so that only (or primarily) target tissue or cells are treated. Cells and tissue with longer membrane charging time constants may require longer (e.g., larger packet sizes) to achieve the treatment threshold as compared with tissue/cells having a shorter time constant. Even when the total energy applied is about the same, different cell and tissue types may respond differently based on their membrane charging time constant.

### Examples

The methods and apparatuses described herein may be used to treat tumors. In one example tumors were generated in mouse skin by injecting 200,000 tumor cells intradermally into the dorsal skin and waiting 6 days for the tumors to grow to approximately 4 mm in diameter. The dorsal skin is easily pulled away from the body of the mouse and stretched over a silicone column with light shining through it to reveal the tumor outline by transillumination. Two parallel rows of needle electrodes were positioned 5 mm apart around the tumor without affecting other body organs. FIGS. 2A-2D illustrate the example of a tumor grown as described herein. FIG. 2A shows the skin region with the tumor before treatment, FIG. 2B shows the same region immediately after treatment, FIG. 2C shows the same region 3 days after treatment, and FIG. 2D shows the same region 25 days after the treatment was applied.

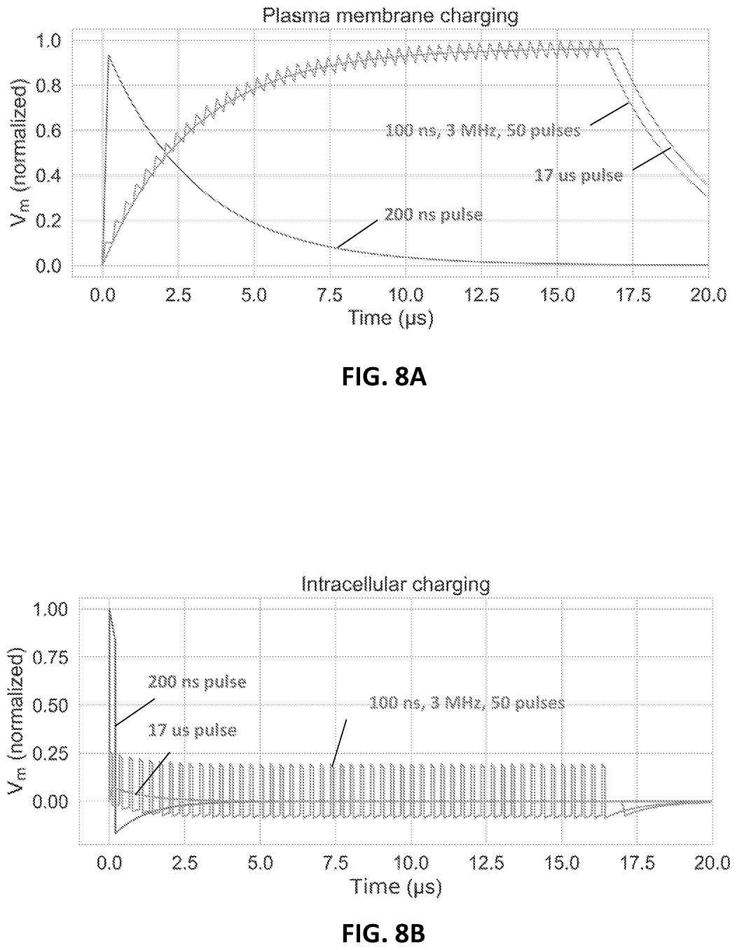

These model tumors were used to test the effect of the application of packets of sub-microsecond, MHz (e.g., 0.5 MHz or greater) pulses on various tumor tissues. In some cases (see, e.g., FIGS. 8A-8B, below) plasma and intracellular membrane charging estimates were calculated. For example, plasma membrane was assumed as a perfect capacitor charging through a resistor with a given RC charging time constant. The intracellular membrane charging with different electrical pulse exposures was calculated using a commonly-utilized model circuit from the literature. The model circuit was tested with MacSpice circuit simulator (Version 3.1.25 (343)). All potentials were normalized to the maximum value in the set.

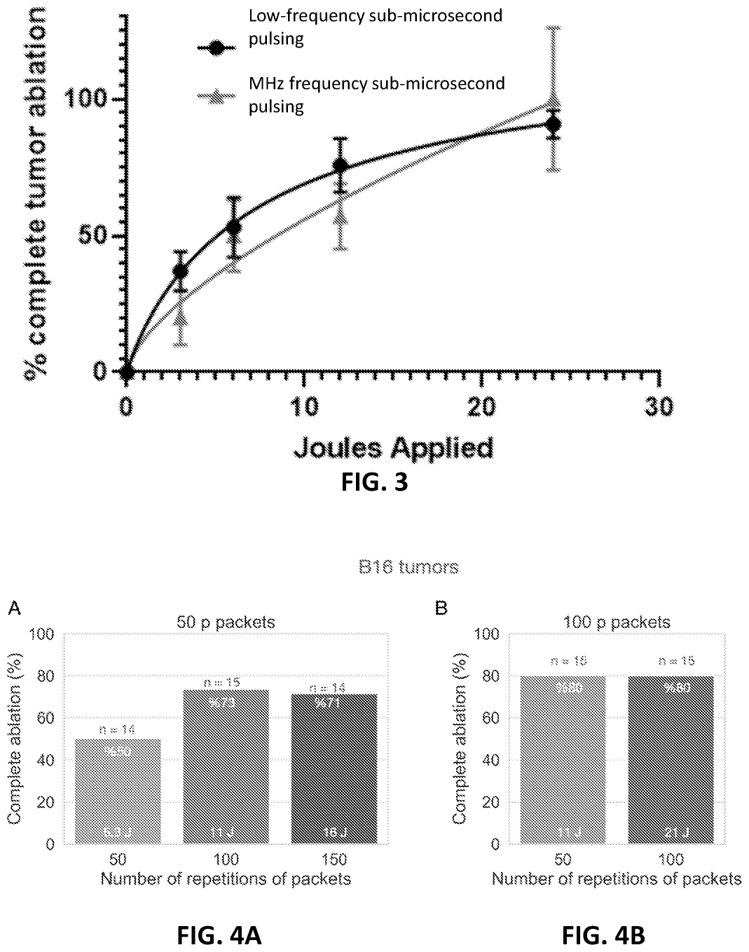

Experimentally, the use of sub-microsecond (e.g., nanosecond) pulse at high frequency (e.g., >0.5 MHz, such as 3 MHz) were examined for different sizes of packets (e.g., different numbers of pulses), as will be described in FIGS. 3, 4A-4D, 5A-5E, 7A-7F and 9A-9B.

FIG. 3 shows a graph summarizing initial results for treating (e.g., killing) B16 tumors using large (e.g., 1000 pulse) packets. In this example, comparable tumor ablation was seen when comparing more traditional sub-microsecond treatment (“low-frequency sub-microsecond pulsing”) with MHz frequency sub-microsecond pulsing. For example, 100 ns, 3 MHz, 1000-pulse (1000 p) MHz packets were used to treat B16 tumors at 8 kV/cm. These results showed that the treatment outcomes changed with energy in a similar manner as it does with more traditional sub-microsecond (e.g., nanosecond) pulse treatments of monophasic 200 ns pulse widths, delivered at 6-8 Hz rate.

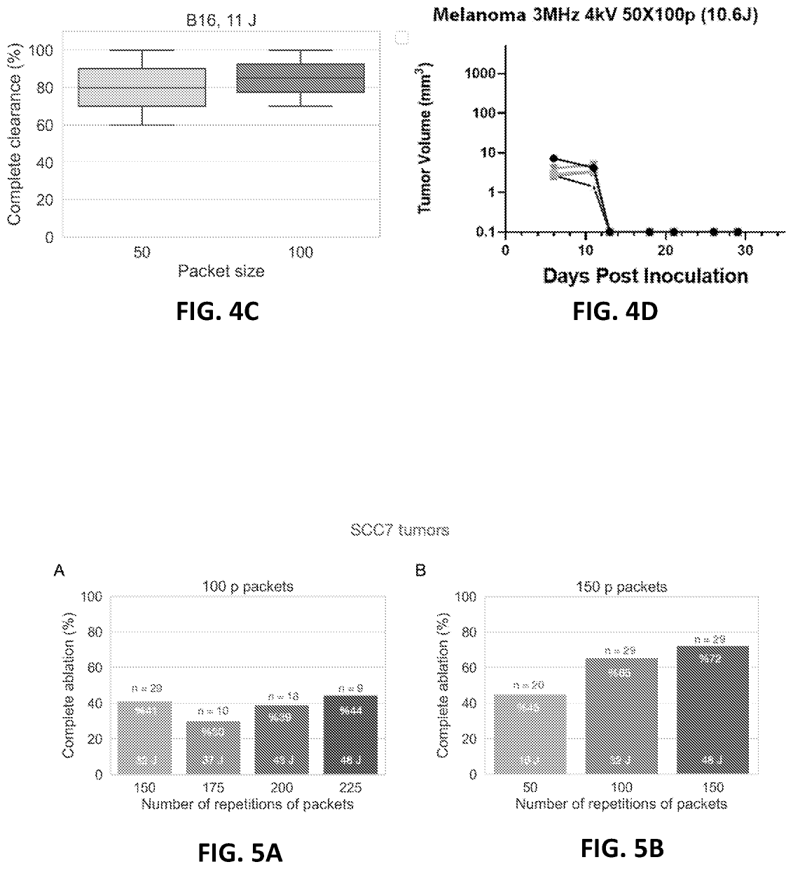

FIGS. 4A-4B illustrate the effectiveness of treatment (e.g., as percentage of complete ablation) for model B16 melanoma tumors when treated with either 50 pulses per packet or 100 pulses per packet when repeating the application of the packets to the tissue for different numbers of repetitions (corresponding to different energy levels). For example, in FIG. 4A, packets having 50 pulses per packet were applied either 50, 100 or 150 repetitions (e.g., providing in 5.3 J, 11 J or 16 J). Packets were repeated at 3 Hz. Increasing the number of packets delivered from 50 to 100 resulted in a significant increase in the percentage of complete ablation (from 50% to 73%). Surprisingly, further increasing the total number of packets delivered did not further improve the percentage of compete ablation of the B16 tumors. However, increasing the size of the packet to 100 pulses, as shown in FIG. 4B resulted much higher percentages of complete ablation that was equivalent for both 50 total packets delivered (11 J, 80%) and 100 total packets delivered (21 J, 80%). Thus, B16 tumors can be ablated with about 50 pulse and 100 pulse packets at a lower total energy level of 11 J.

FIG. 4C illustrates a comparison between B16 tumors treated with equivalent energy (11 J) and different packet sizes (e.g., 50 packets or 100 packets). The total energy applied may be adjusted by increasing or decreasing the total number of packets delivered in a treatment. In this case, equal energy treatment was delivered with 50 pulse or 100 pulse packets. FIG. 4D illustrates the effect of delivering packets of 100 pulses of a high-frequency (e.g., 3 MHz), sub-microsecond pulses (at 11 J) on B16 tumors, showing a nearly compete reduction in tumor volume following treatment in all animals tested in one of the experiments.

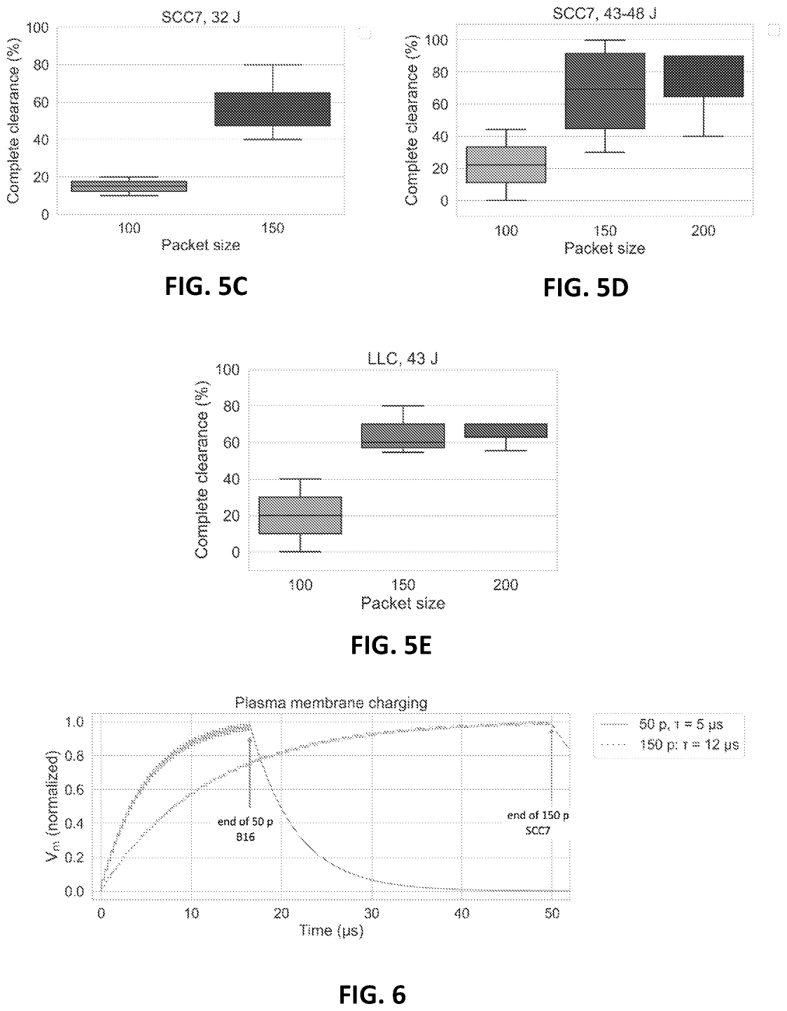

Similar results with different packet size thresholds for effective treatment were found with other tissue/cell types. For example, FIGS. 5A-5D show results with SCC7 tumors and FIG. 5E shows similar results with LLC tumors. As shown in FIGS. 5A and 5B, treatments with packets of sub-microsecond, MHz pulses were not consistently successful up to 50 J with packets of 100 pulses (FIG. 5A) even when increasing the total number of packets delivered (and therefore the energy applied). However, as shown in FIG. 5B, with 150 pulse (150 p) packets even at lower energy (e.g., 32 J), higher ablation rates were seen than with 100 pulse packets.

FIG. 5C shows the percentage of complete clearance (e.g., ablation) in SCC7 cells when 32 J of energy are applied by different sized packets. Minimal effects were seen when the packet size was 100 pulses, wherein increasing the packet size to 150 pulses, even while the total energy delivered is kept constant at 32 J resulted in a significant increase in the percentage of tumors showing complete clearance. FIG. 5D shows similar effects for packets of 100 pulses, 150 pulses or 200 pulses, in which the total energy applied was between 43-48 J. Both 150 and 200 pulse packets showed a significant percentage of complete clearance as compared with 100 pulse packets.

LCC tumor cells were also examined, and the results are summarized in FIG. 5E. In this example, the total energy was maintained at approximately 43 J, and the percentage of complete clearance at packets having 100 pulses was lower than the percentage at both 150 and 200 pulses per packet. Thus, effective clearance of SCC and LLC tumors in these examples typically required packets of 150 pulses or more when pulsed within the packet at 3 MHz packets.

These findings indicate that at the same energy levels, sub-microsecond pulses at MHz frequency in packet sizes exceeding a critical level (e.g., 50 p for B16 and 150 p for SCC7 and LCC tumors) determined the treatment success. This appears to be based on charging time constant of the tumor type, allowing for the application of lower total energy to accomplish a significant therapeutic effect. Without being bound by a particular theory, at lower packet sizes, membrane charging may not have enough time to reach the required levels for successful ablation, but that level may be reached above a certain packet size. For B16 tumors, assuming a 3 MHz pulsing rate within the packet, and assuming that a 50 p packet corresponds to about three times the charging time constant, τ, the resulting membrane charging time constant (τ) is likely ˜5-8 μs. For SCC7, the same calculation leads to a membrane charging time constant (τ) of about ˜12-15 μs. A theoretical picture of membrane charging for both cases is shown in FIG. 6. FIG. 6 shows a theoretical calculation of plasma membrane charging with B16 and SCC7 tumors based on complete ablation results obtained with 3 MHz packets of different sizes shown above. The example shown in FIG. 6 illustrates that time-constant based tuning of packet size can be used to target tumor tissue and spare surrounding normal tissue, since cancerous tissues, in general, have a lower time constant than their normal counterparts, which may be at least in part due to their higher water content and higher conductivity.

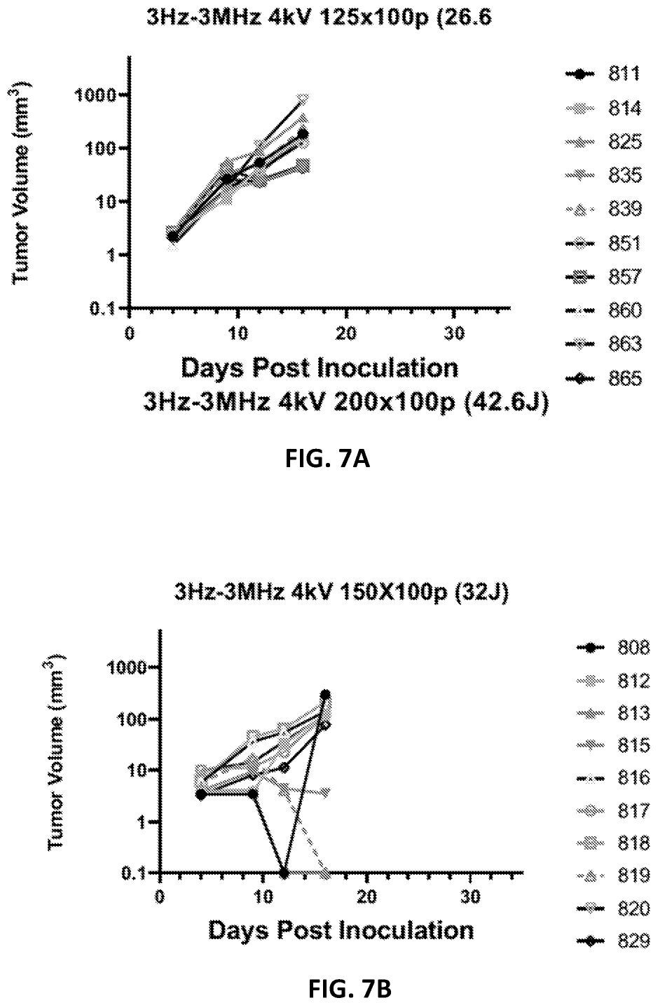

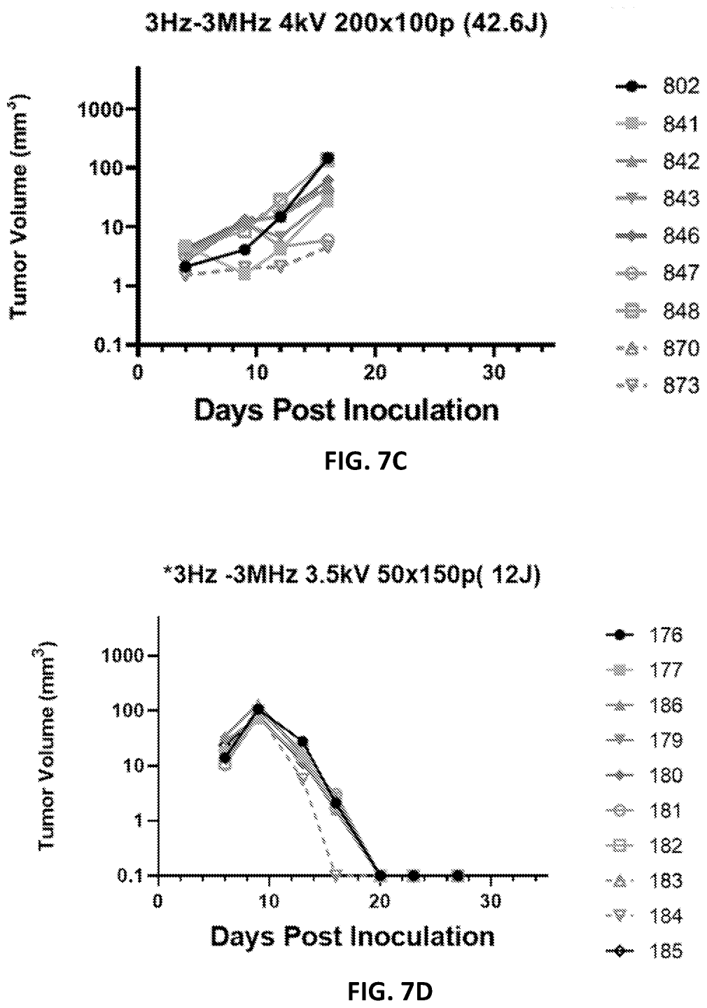

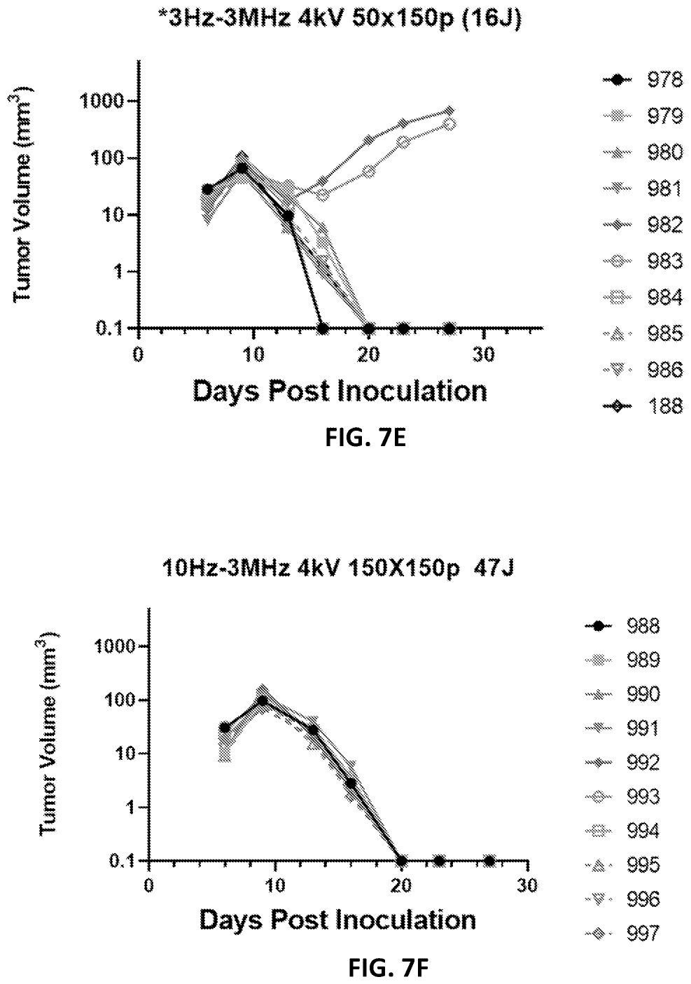

FIGS. 7A-7C and 7D-7F show the results of SCC tumor clearance as measured in experiments with packets of sub-microsecond, 3 MHz pulses applied at the same energy level for either 100 pulse (FIGS. 7A-7C) or 150 pulse (FIGS. 7D-7F) packets. The larger packets (e.g., 150 pulses) shown in FIGS. 7D-7F are significantly better at ablating the tumors than the similar or higher-energy smaller sized packets shown in FIGS. 7A-7C. Surprisingly, additional experiments found that there was no further significant increase beyond 150 pulse packets.

The rapid-fire sequence of pulses in the sub-microsecond, high frequency (e.g., MHz) energy applied may not allow substantial discharging between the individual pulses. A theoretical plasma membrane potential charging curve may look almost identical to that of an equivalent microsecond duration pulse, as shown in FIG. 8A. The equivalence can be established analytically and corresponds to a larger, microsecond, pulse of the same duration as the whole MHz packet but is scaled in amplitude by the duty cycle of the MHz train (shown as the smooth trace in FIG. 8A). An important difference between the two exposures, however, is the rapid charging and discharging of the intracellular membranes due to the high-frequency components of sub-microsecond, 3 MHz packets, which is absent in the microsecond pulse exposures, as shown in FIG. 8B. FIGS. 8A and 8B show the relative theoretical charging of plasma and intracellular membranes by traditional sub-microsecond pulsing (e.g., 200 ns, 30 kV/cm), compared to sub-microsecond pulsing at high frequencies (e.g., 3 MHz) in a packet of 50 pulses (100 ns, 8 kV/cm), and compared to an equivalent microsecond pulse exposure (17 μs, 2.4 kV/cm).

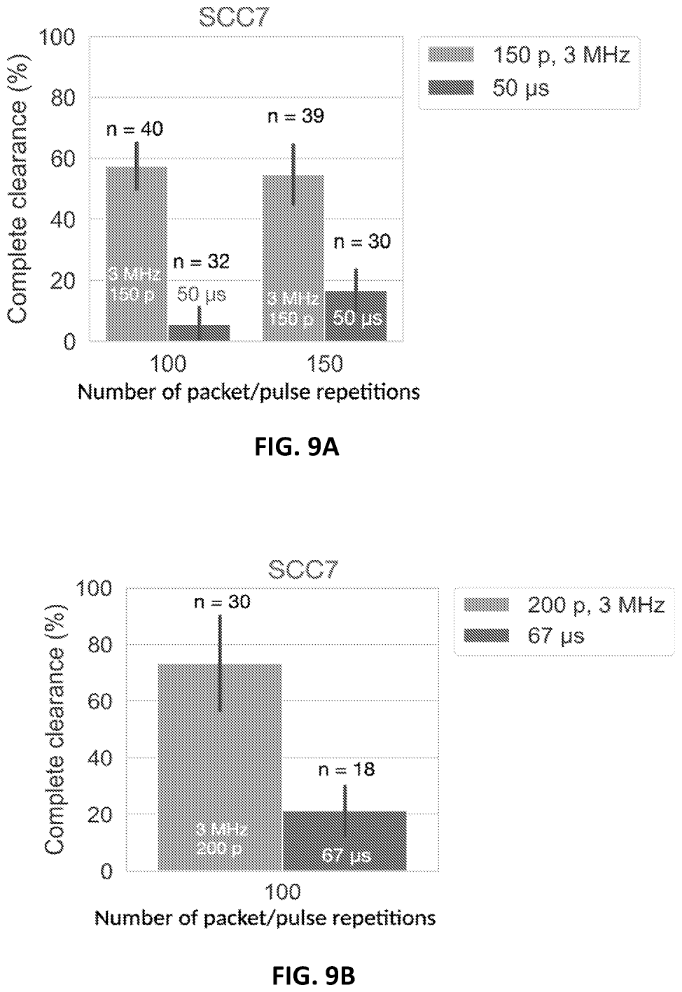

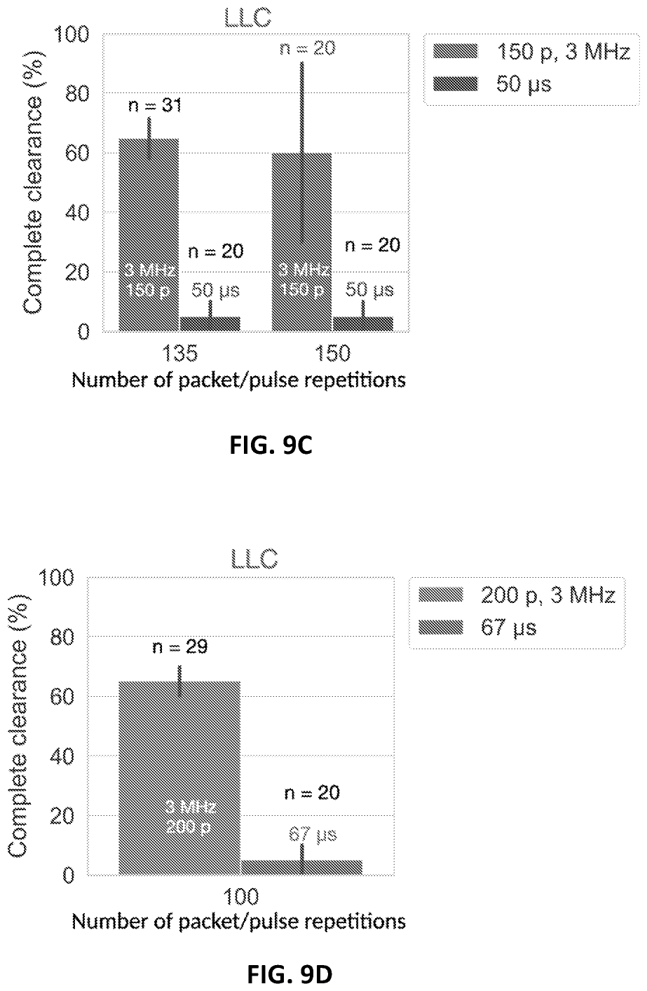

The improved treatment effect when matching packet size (e.g., number of pulses in a packet) to the membrane charging time constant does not appear to be due to membrane charging. In vivo experiments with SCC7 and LLC tumors were performed to examine clearance endpoint and of cellular membrane charging in tissues. 3 MHz nanosecond pulse packets were compared to their charge-equivalent μs pulse exposures. These results are shown in FIGS. 9A-9D. In all experiments, μs pulse exposures of equivalent charge were significantly less efficient than their MHz counterparts. This difference may be partly due to the high-frequency components of MHz nanosecond pulse that are absent in μs pulses.

Thus, the ablation response between sub-microsecond, high frequency (e.g., >0.5 MHz) packets and equivalent microsecond duration pulse exposures show that sub-microsecond, high frequency (e.g., 3 MHz) packets were much more effective at ablating SCC7 tumors than the equivalent microsecond pulse (FIGS. 9A-9B). This suggests that the perturbation of the intracellular membranes may also contribute to the ablation efficacy. In FIG. 9A SCC7 tumor tissue was ablated either with pulse packets having 150 sub-microsecond pulses at 3 MHz or with a 50 μs pulse, where each of the packet or the 50 μs pulse were repeated for either 100 or 150 repetitions at 3 Hz. In both cases, the sub-microsecond, high frequency (3 MHz) packets of equivalent size and amplitude to the 50 μs pulse and were significantly more effective in tumor ablation as compared to their microsecond pulse counterparts. Similarly, FIG. 9B shows an example in which packets of 200 sub-microsecond, 3 MHz pulses were compared with 67 μs pulses that are each applied for 100 repetitions of either the packet or the 67 μs pulse. Microsecond-long pulse exposures with equivalent capacitive charging as these MHz packets are significantly less efficient in tumor clearance. The expected single but high amplitude intracellular charging effect with common sub-microsecond energy application at lower frequencies may lead to biologically distinct effects as compared with sub-microsecond, high frequency packet energy application.

FIGS. 9C and 9D show similar results for LCC tumors. In FIG. 9C LLC tumor tissue was ablated either with pulse packets of 150 sub-microsecond pulses at 3 MHz or with a 50 μs (single) pulse, where each of the packet or the 50 μs pulse were repeated for either 135 or 150 repetitions at 3 MHz. In both cases, the sub-microsecond, high frequency (3 MHz) packets of equivalent size and amplitude to the 50 μs pulse were significantly more effective in tumor ablation as compared to their microsecond pulse counterparts. Similarly, FIG. 9D shows an example in which packets of 200 sub-microsecond, 3 MHz pulses were compared with a single 67 μs pulse in which each were applied for 100 repetitions of either the packet or of the 67 μs pulse. Thus, tumor clearance of the MHz packets of nanosecond (sub-microsecond) pulses was significantly better as compared to charge-equivalent microsecond pulses for both SCC7 and LLC tumors. Thus, MHz sub-microsecond packets of pulses are more effective than their charge-equivalent counterparts. This may be due to the high-frequency components of MHz packets affecting intracellular charging as well as cellular and extracellular charging.

### Apparatus

An apparatus for specifically treating a target tissue using sub-microsecond, high frequency (e.g., 0.5 MHz or greater) packets of limited pulse number may include a pulse generator, an applicator coupled to the pulse generator, and a controller controlling the pulse generator and delivery of energy by the applicator. The controller may include one or more processors, a memory and software/hardware and/or firmware for controlling the apparatus, and in particular, in some examples, for identifying the target (and in some cases non-target) membrane charging time constant and adjusting or setting the number of pulses in the packet according to the identified membrane charging time constant. The controller may be a single processor or a series of processors that communicate with each other (wireless or by wired connection). In some examples the controller may be integrated with the pulse generator. Alternatively, the controller may be coupled with the pulse generator. In some examples the controller may be software that is run on a remote (e.g., cloud-based) server and that communicates and/or commands the pulse generator, including setting the packet size based on the determination of the target (and in some cases the non-target) membrane charging time constant.

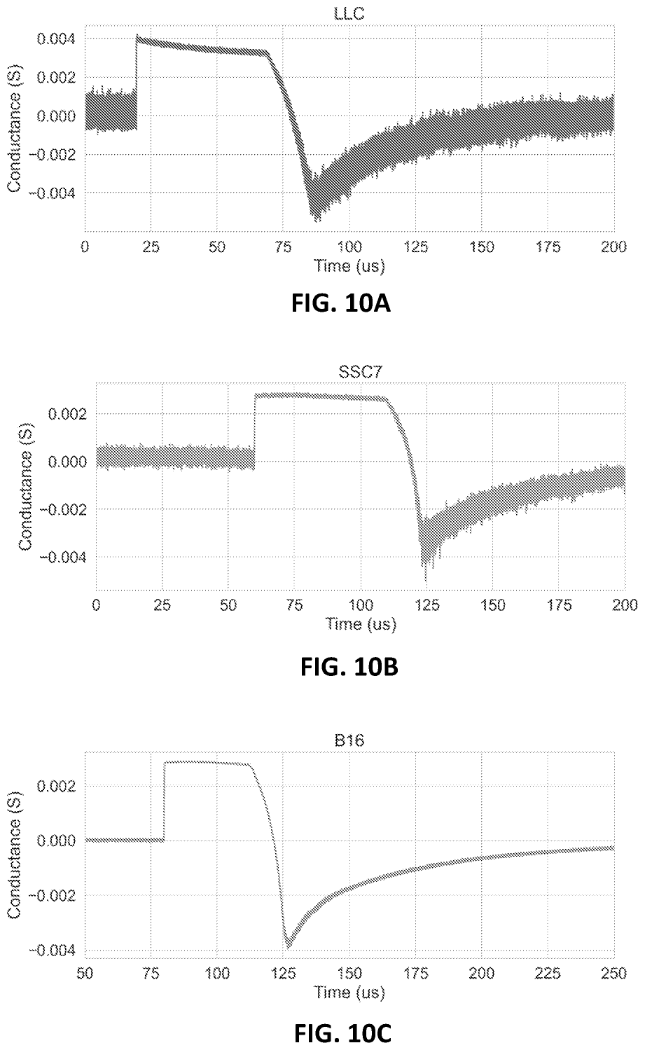

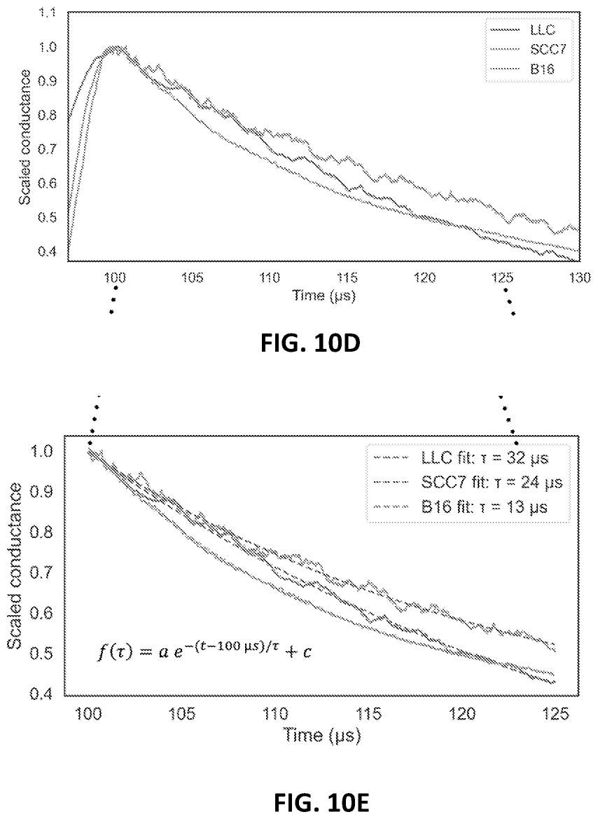

Any of the apparatuses described herein may be configured to automatically or semi-automatically estimate the appropriate packet size using a microsecond test pulse waveform. For example, test pulses may be used to estimate the packet sizes for a given tissue using microsecond pulse exposure waveforms. In one example, microsecond pulse exposure waveforms were applied to B16, SCC7, and LLC tumors, and conductance values for each tumor tissue type were calculated by taking the ratio of current to voltage waveforms measured. The conductance with time may be determined by first determining or estimating an offset the voltage waveforms sufficient to move the entire waveform above a zero level (e.g., to avoid division by zero artifacts), and the ratio of current to voltage may be measured during exposure. Examples of the conductance change with time are shown in FIGS. 10A-10C for each of three tissues, e.g., LLC tumor tissue (FIG. 10A), SSC7 tissue (FIG. 10B), and B16 tumor tissue (FIG. 10C).

The exponential approach to zero may then be calculated using the conductance waveform following the pulse exposure. FIGS. 10D and 10E illustrate one example of this technique. In this example, B16 waveforms have significantly smaller time constants as compared to LLC and SSC7. As shown in FIG. 10E, exponential fits may be done, e.g., for the first 25 μs after the negative peak of the conductance curves, after the curves were inverted and filtered for noise. A fitting function, such as shown in Equation (1), below:

$$f ( τ ) = a {e}^{( - ( t - 100 μ s ) / τ )} + c \qquad ( Equation 1 )$$

where a and c are arbitrary constants and τ is the time constant, may be used. The resulting τ values (LLC τ is 32 μs, SCC7 τ is 24 μs, and B16 τ is 13 μs) identified by the exponential fits for the three tissues are also shown in FIG. 10E. The results of these fits and the comparison show that the time constants of B16, SCC7, and LLC determined using a test pulse are in increasing order: 13 μs for B16, 24 μs for SCC7, and 32 μs for LLC, and are in good agreement with published values. Any of the methods and apparatuses described herein may use similar measurements using low-energy, non-ablating microsecond pulses for pre-treatment estimation of packet size for different tissues. In some examples a database of the pre-measured conductance for different tissues can also or alternatively be incorporated into the device software.

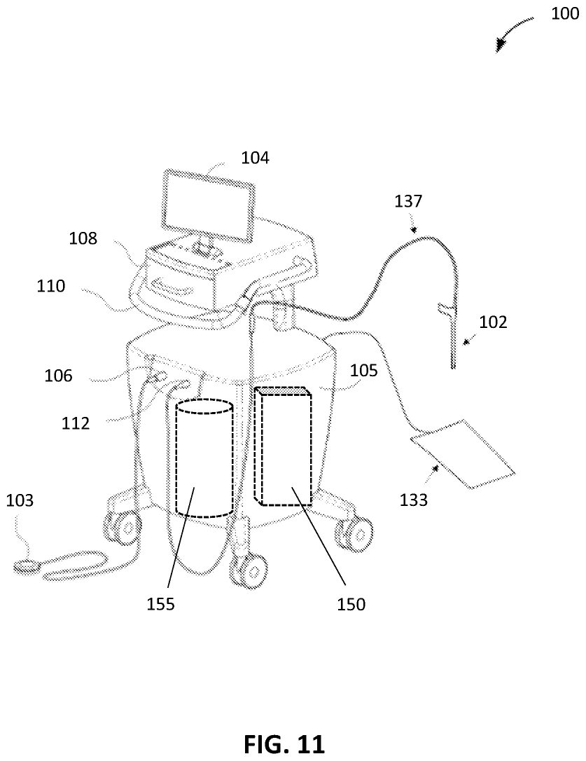

For example, FIG. 11 illustrates one example of an apparatus (e.g., system or device) 100 including a pulse generator 155, applicator 102 and controller 150. The pulse generator 155 is shown within cabinet 105, as is the controller 150. The controller and pulse generator may be connected to the various components (not shown) as well as each other. The pulse generator is configured for delivering fast (e.g., sub-microsecond) pulses of electrical energy. The apparatus may include an applicator 102 (shown by example as an elongate, hand-held applicator tool) that may include one or more removably and/or disposable tips (not shown) for applying energy. The apparatus may include one or more user controls or interfaces, including a footswitch 103, and manual user interface 104 (e.g., monitor such as a touchscreen). Footswitch 103 is connected to housing 105 (which may enclose the electronic components) through a cable and connector 106. The applicator 102 may include electrodes or may couple to a removable tip with electrodes and may be connected to housing 105 and the electronic components therein through a cable 137 and high voltage connector 112. The apparatus 100 may also include a handle 110 and storage drawer 108. The apparatus 100 may also include a holder (e.g., holster, carrier, etc.) (not shown) which may be configured to hold the applicator 102. In some examples the system may be configured for monopolar treatment and may optionally include a dispersive electrode 133 (e.g., a return electrode pad).

In use, a human operator may control the operation of the device (on/off, apply pulses/packet, etc.) and may input/output control information into the apparatus using one or more inputs. In particular, the user may enter information about the target tissue, which may be used to determine the membrane charging time constant, such as the tissue type (e.g., tumor name/category), size, shape, etc. In some examples the apparatus may include a bioimpedance module for measuring or estimating bioimpedance from the target and/or non-target tissues. The controller may determine or estimate a membrane charging time constant from this input. In some examples the apparatus may include one or more menus, including a menu of possible tumor types to be treated, and may have a corresponding database of time constants for selected tumor types (and/or sizes, etc.).

The apparatus may automatically or semi-automatically, or manually select the number of pulses (packet size), amplitude, pulse duration, and frequency information to be applied based on the determined time constant(s). The controller (for example, through one or more processors) may then set the pulse generator to generate the determined parameters and may control the delivery/application of the pulse packet from the applicator. The applicator 102 may be hand-held (e.g., by a user) or it can be affixed to or part of a movable arm of a robotic system, and its operation may be at least partially automated or fully automated, including computer-controlled operation.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor, causes the processor to perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An apparatus for specifically ablating a target tissue, the apparatus comprising:
a pulse generator;
an applicator configured to apply electrical energy from the pulse generator to two or more electrodes; and
a controller comprising one or more processors and a memory storing computer-program instructions, that, when executed by the one or more processors, perform a computer-implemented method comprising:
setting a packet size of a packet of sub-microsecond pulsed electrical energy having a frequency of greater than 0.5 MHz based on an identified membrane charging time constant for the target tissue or cells; and
applying, from the pulse generator, the packet of sub-microsecond pulsed electrical energy between the two or more electrodes, when triggered by a user input, to selectively and specifically kill the target tissue.

2. The apparatus of claim 1, wherein the computer-implemented method further comprises identifying the membrane charging time constant for the target tissue or cells.

3. The apparatus of claim 2, wherein the computer-implemented method includes receiving a description of the target tissue or cells and wherein identifying the membrane charging time constant comprises identifying the membrane charging time constant for the target tissue or cells based on the description.

4. The apparatus of claim 2, wherein receiving the description comprises receiving one or more of: a tissue type of the target tissue or cells, a cell size of the target tissue or cells, a cell shape of the target tissue or cells, a fat content of the target tissue or cells, and a water content of the target tissue or cells.

5. The apparatus of claim 1, wherein the computer-implemented method is configured to identify the membrane charging time constant by determining a fat and/or water content of the target tissue or cells and estimating the membrane charging time constant from the fat and/or water content.

6. The apparatus of claim 1, wherein the controller is further configured to determine a bioimpedance measurement from the two or more electrodes and wherein the computer-program instructions further comprise identifying the membrane charging time constant for the target tissue or cells from the bioimpedance measurement.

7. The apparatus of claim 1, wherein the computer-implemented method further comprises identifying a membrane charging time constant for a non-target tissue or cells adjacent to the target tissue or cells.

8. The apparatus of claim 1, wherein the computer-implemented method is configured to limit a total number of pulses in the packet of sub-microsecond pulsed electrical energy to target tissue or cells having a sufficiently low time constant.

9. The apparatus of claim 1, wherein the computer-implemented method is configured to target a tumor tissue and spare a surrounding normal tissue by tuning the packet size.

10. The apparatus of claim 1, wherein the two or more electrodes comprise an array of needle electrodes.

11. The apparatus of claim 1, wherein the computer-implemented method is configured to set the packet size of the packet of sub-microsecond pulsed electrical energy so that the sub-microsecond pulsed electrical energy comprises sub-microsecond pulses having a pulse duration of between about 1 ns to about 1000 ns.

12. The apparatus of claim 1, wherein the computer-implemented method is configured to set the packet size of the packet of sub-microsecond pulsed electrical energy comprises sub-microsecond pulses having a pulse amplitude of between about 1 kV/cm to about 12 kV/cm.

13. The apparatus of claim 1, wherein the computer-implemented method is configured to set the packet size of the packet of sub-microsecond pulsed electrical energy so that the packet size comprises between about 30 and 300 pulses.

14. The apparatus of claim 1, wherein the controller is configured to identify the membrane charging time constant by applying a low-energy test pulse and determining a time constant from a time course of a conductance from the test pulse.

15. An apparatus for specifically ablating a target tissue, the apparatus comprising:
a pulse generator;
an applicator configured to apply electrical energy from the pulse generator to two or more electrodes; and
a controller comprising one or more processors and a memory storing computer-program instructions, that, when executed by the one or more processors, perform a computer-implemented method comprising:
identifying a membrane charging time constant for the target tissue;
setting a packet size of a packet of sub-microsecond pulsed electrical energy having a frequency of between 0.5 MHz and 5 MHz based on the identified membrane charging time constant; and
applying, from the applicator, the packet of sub-microsecond pulsed electrical energy between the two or more electrodes to selectively and specifically kill the target tissue.

16. A method of specifically killing a target tissue or cells, the method comprising:
setting a packet size of a packet of sub-microsecond pulsed electrical energy having a frequency of greater than 0.5 MHz based on an identified membrane charging time constant for the target tissue or cells;
positioning the target tissue or cells between two or more electrodes; and
killing at least some of the target tissue or cells by applying the packet of sub-microsecond pulsed electrical energy between the two or more electrodes.

17. The method of claim 16, further comprising identifying the membrane charging time constant for the target tissue or cells.

18. The method of claim 16, further comprising identifying the membrane charging time constant by receiving a description of the target tissue or cells and looking up the membrane charging time constant based on the description.

19. The method of claim 16, further comprising identifying the membrane charging time constant by determining a bioimpedance measurement from the target tissue or cells and estimating the membrane charging time constant for the target tissue from the bioimpedance measurement.

20. The method of claim 16, further comprising identifying the membrane charging time constant by determining a fat and/or water content of the target tissue or cells and estimating the membrane charging time constant from the fat and/or water content.

21. The method of claim 20, further comprising identifying the membrane charging time constant by receiving a description of the target tissue or cells comprising one or more of: a tissue type of the target tissue or cells, a cell size of the target tissue or cells, a cell shape of the target tissue or cells, a fat content of the target tissue or cells, and a water content of the target tissue or cells, and determining the membrane charging time constant based on the description.

22. The method of claim 16, further comprising identifying the membrane charging time constant by estimating the membrane charging time constant from a biopsy of the target tissue.

23.-38. (canceled)
